(19) United States
(12) Patent Application Publication
(10) Pub. No.: US 2014/0206969 A1
(43) Pub. Date: Jul. 24, 2014

(54) BLOOD GLUCOSE METER

(71) Applicant: Samsung Electronics Co., Ltd., Suwon-si (KR)

(72) Inventors: Su-Ho LEE, Suwon-si (KR); Jae-Hong KIM, Incheon (KR); Jae-Geol CHO, Yongin-si (KR); Chul-Ho CHO, Seongnam-si (KR)

(73) Assignee: Samsung Electronics Co., Ltd., Suwon-si (KR)

(21) Appl. No.: 14/154,515

(22) Filed: Jan. 14, 2014

(30) Foreign Application Priority Data
Jan. 23, 2013 (KR) 10-2013-0007601

(51) Int. Cl. A61B 5/145; A61B 5/15

(52) U.S. Cl. CPC A61B 5/14532; A61B 5/1411
USPC 600/365

(57) ABSTRACT

A blood glucose meter is provided. The blood glucose meter includes a body of the blood glucose meter, a lancing unit which is provided at one side of the body of the blood glucose meter and includes a lancet to puncture a skin in order to sample blood of a user, a sensor strip unit which is provided at another side of the body of the blood glucose meter which is different than the one side of the body at which the lancing unit is provided, the sensor strip unit being configured to sample the blood of the user, and a measurement unit which measures the blood sampled by the sensor strip unit. The blood glucose meter has the measurement unit which is integrally assembled with the body of the blood glucose meter and includes the lancing unit having the lancet and the sensor strip unit.

### CROSS-REFERENCE TO RELATED APPLICATION(S)

This application claims the benefit under 35 U.S.C. §119(a) of a Korean patent application filed on Jan. 23, 2013 in the Korean Intellectual Property Office and assigned Serial number 10-2013-0007601, the entire disclosure of which is hereby incorporated by reference.

### TECHNICAL FIELD

The present disclosure relates to a blood glucose meter. More particularly, the present disclosure relates to a blood glucose meter in which a lancing unit with a lancet is integrated with a measurement unit having a sensor strip unit.

### BACKGROUND ART

Generally, with a diagnosis and treatment of diabetes, a necessity for periodic measurements of a concentration of blood glucose in blood has been increased. Such a blood glucose measurement can be easily carried out by using a blood glucose meter. A blood glucose meter takes a blood sample from a patient by using a bio-sensor in the form of a sensor strip, and then measures a blood glucose level by using an electric signal generated through an electrochemical reaction of the taken blood and a chemical substance in the bio-sensor.

A patient having a disease such as diabetes is required to periodically measure a concentration of blood glucose in the blood. Accordingly, there is a necessity for a portable blood glucose meter, a sensor strip, a lacing unit, a lancet and cotton. The necessity for cotton may be substituted with a washing of a portion at which a blood sample is taken.

According to the related art, a user has inconvenience in always carrying the above-mentioned units in order to measure the blood glucose. According to the related art, the measurement cannot be performed when any one of the units is not present, thereby inconveniencing a user.

Further, when a user measures the blood glucose, there are facts causing various errors. Most of the facts are caused by the user. For example, when the user puts lotion on the user's hands or takes out a sensor strip from a container with a hand used to eat food, the sensor trip may be polluted by holding a blood inlet of the sensor strip. Similarly, one of errors caused by the user is that the sensor strip is abnormally inserted into the blood glucose meter. Moreover, in the blood glucose meter according to the related art, introducing blood into a sensor strip may be further complicated because of a lancing unit in the case that the lancing unit and the sensor strip are present together at a side of the blood glucose meter. In addition, a disadvantage of the blood glucose meter according to the related art may be that blood pollutes the lancing unit.

Therefore, in order to overcome the disadvantages, it is necessary to integrate a lancet for taking blood, a lancing unit, a sensor strip, and a blood glucose meter in one piece and also a device capable of separating the lancing unit and the sensor strip from each other is required.

The above information is presented as background information only to assist with an understanding of the present disclosure. No determination has been made, and no assertion is made, as to whether any of the above might be applicable as prior art with regard to the present disclosure.

### SUMMARY

Aspects of the present disclosure are to address at least the above-mentioned problems and/or disadvantages and to provide at least the advantages described below. Accordingly, an aspect of the present disclosure is to provide a blood glucose meter in which a lancing unit with a lancet and a sensor strip unit are integrally assembled so as to remove an inconvenience of always carrying units such as a lancet, a lancing unit, a sensor strip, and a measurement unit, and which may be easily used with a hand of a user, thereby improving utility thereof

Another aspect of the present disclosure is to provide a blood glucose meter in which as a cover of a sensor strip unit is slidably moved, a sensor strip is made to be exposed out of the blood glucose meter to measure a blood glucose level so that a user need not put out the sensor strip from a container with a hand holding it, thereby previously preventing the sensor strip from being polluted by the user's hand.

Another aspect of the present disclosure is to provide a blood glucose meter which includes a lancing unit with a lancet for taking blood at one side thereof, and a measurement unit with a sensor strip reacting with blood at the other side thereof, thereby removing an inconvenience caused by the blood glucose meter according to the related art in which a lancing unit and a sensor strip unit are arranged together at a side thereof, accurately introducing blood into the sensor strip, and preventing the lancing unit from being polluted by the blood.

In accordance with an aspect of the present disclosure, a blood glucose meter is provided. The blood glucose meter includes a measurement unit integrally assembled with a body of the blood glucose meter, and including a lancing unit having a lancet and a sensor strip unit.

In accordance with another aspect of the present disclosure, a blood glucose meter is provided. The blood glucose meter includes a body of the blood glucose meter, a lancing unit which is provided at one side of the body of the blood glucose meter and includes a lancet to puncture a skin in order to sample blood of a user, a sensor strip unit which is provided at another side of the body of the blood glucose meter which is different than the one side of the body at which the lancing unit is provided, the sensor strip unit being configured to sample the blood of the user, and a measurement unit which measures the blood sampled by the sensor strip unit.

In accordance with another aspect of the present disclosure, a blood glucose meter is provided. The blood glucose meter includes a body of the blood glucose meter, a lancing unit configured to puncture a skin of a user, a sensor strip unit comprising a plurality of sensor strips, the sensor strip unit configured to discharge one of the plurality of sensor strips in order to sample the blood of the user, and a measurement unit configured to measure the sampled blood of the user.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is an exploded view illustrating a lancing unit of a blood glucose meter according to an embodiment of the present disclosure;

FIG. 2 is a sectional view illustrating an assembly of a lancing unit of a blood glucose meter according to an embodiment of the present disclosure;

FIG. 3 is a sectional view illustrating a lancing unit of a blood glucose meter before operation of the lancing unit according to an embodiment of the present disclosure;

FIG. 4 is a sectional view illustrating an operational state of a lancing unit of a blood glucose meter according to an embodiment of the present disclosure;

FIG. 5 is an exploded perspective view illustrating a sensor strip unit of a blood glucose meter according to an embodiment of the present disclosure;

FIG. 6 is a perspective view illustrating an assembly of a sensor strip unit of a blood glucose meter according to an embodiment of the present disclosure;

FIG. 7 is a perspective view illustrating an assembly of a sensor strip unit and a strip cartridge of a blood glucose meter according to an embodiment of the present disclosure;

FIG. 8 is a side view illustrating a strip cartridge mounted on a blood glucose meter according to an embodiment of the present disclosure;

FIG. 9 is a side view illustrating a sensor strip unit of a blood glucose meter in which a cover of the sensor strip unit is slidably moved according to an embodiment of the present disclosure;

FIG. 10 is a sectional view illustrating a sensor strip unit of a blood glucose meter before a sensor strip operates according to an embodiment of the present disclosure;

FIG. 11 is a sectional view illustrating a sensor strip unit of a blood glucose meter during an operation of sensor strip unit according to an embodiment of the present disclosure;

FIG. 12 is a sectional view illustrating a sensor strip unit of a blood glucose meter after operation of the sensor strip unit according to an embodiment of the present disclosure;

FIG. 13 is a perspective view illustrating a sensor strip of a blood glucose meter in which the sensor strip is exposed to an external environment according to an embodiment of the present disclosure; and

FIG. 14 is a side view illustrating a measurement unit of a blood glucose meter according to an embodiment of the present disclosure.

The same reference numerals are used to represent the same elements throughout the drawings.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the present disclosure. Accordingly, it should be apparent to those skilled in the art that the following description of various embodiments of the present disclosure is provided for illustration purpose only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

With respect to the terms in the various embodiments of the present disclosure, the general terms which are currently and widely used are selected in consideration of functions of structural elements in the various embodiments of the present disclosure. However, meanings of the terms may be changed according to an inventor's intention, a judicial precedent, appearance of a new technology, and the like. Further, in a specific case, the applicant may select terms. In this case, the meanings of the terms will be described in the detailed description of the various embodiments of the present disclosure. Accordingly, the terms used in the various embodiments of the present disclosure should be defined based on not simple names thereof but the meanings thereof and contents throughout the various embodiments of the present disclosure.

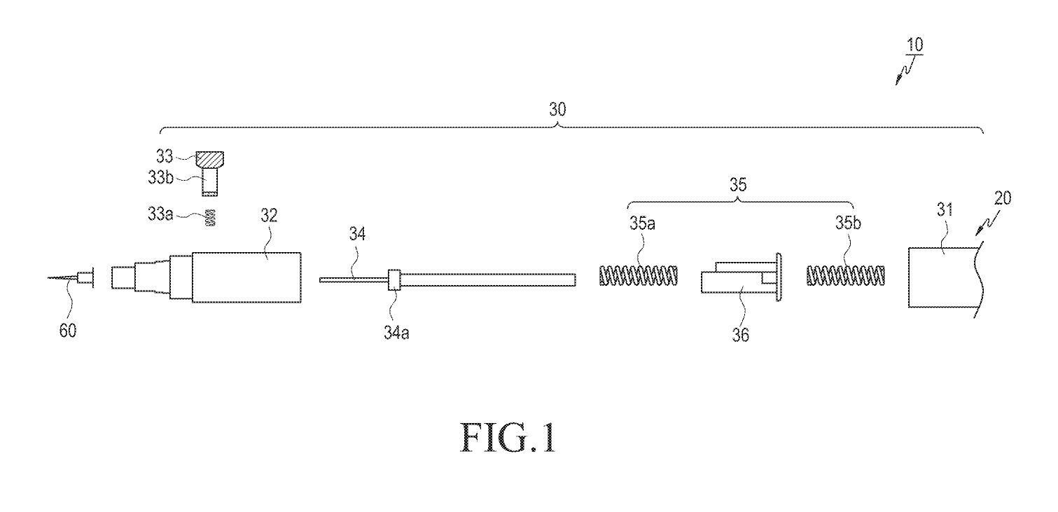

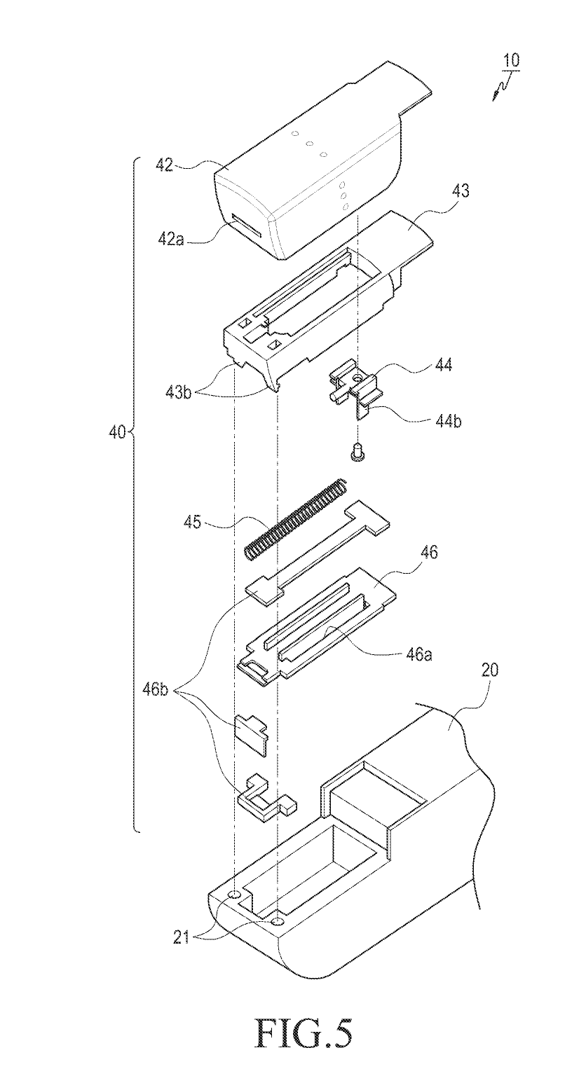

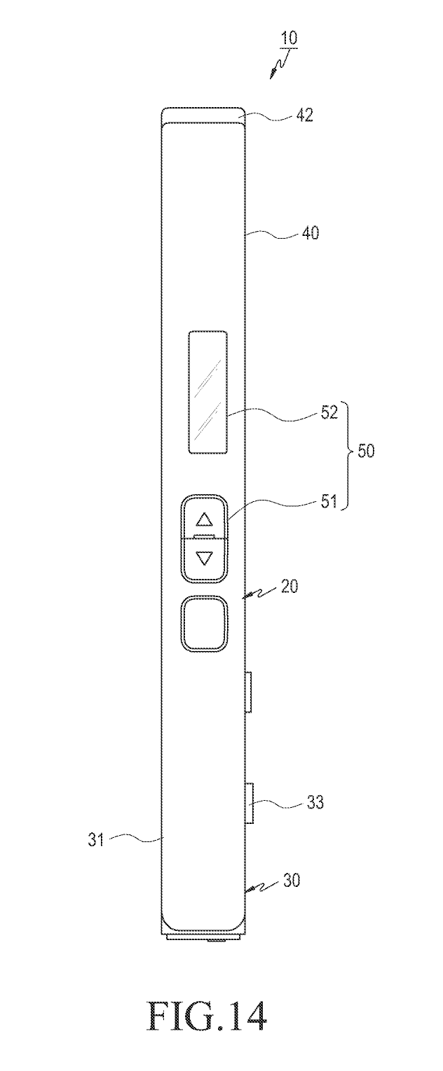

FIG. 1 is an exploded view illustrating a lancing unit of a blood glucose meter according to an embodiment of the present disclosure. FIG. 5 is an exploded perspective view illustrating a sensor strip unit of a blood glucose meter according to an embodiment of the present disclosure. FIG. 14 is a side view illustrating a measurement unit of a blood glucose meter according to an embodiment of the present disclosure.

Referring to FIGS. 1, 5, and 14, a structure of a blood glucose meter will be described. The blood glucose meter 10 includes a body 20, a lancing unit 30 having a lancet 60, a sensor strip unit 40, and a measurement unit 50.

According to the various embodiments of the present disclosure, the body 20 of the blood glucose meter 10 has a pen type shape, and is provided with the lancet 30 at one side thereof and the sensor strip unit 40 at the other side thereof

According to the various embodiments of the present disclosure, the lancing unit 30 is configured to take blood of a user. For example, the lancing unit 30 is configured to cut a skin using the lancet in order to take blood of a user.

According to the various embodiments of the present disclosure, the sensor strip 40 may be configured to receive blood from the lancing unit 30 in order to provide for detection on the blood (e.g., testing and/or measurement of the blood). For example, the sensor strip unit 40 is configured to take the blood.

According to the various embodiments of the present disclosure, the measurement unit 50 is configured to provide detection on the blood (e.g., testing and/or measurement of the blood). For example, the measurement unit 50 measures a glucose level of the blood taken by the sensor strip unit 40.

As described above, the blood glucose meter 10 has a structure in that the lancing unit 30 including the lancet 60 is integrated with the measurement unit 50 having the sensor strip unit 40. Accordingly, easily measuring the glucose level in the blood by using the integrated blood glucose meter is possible.

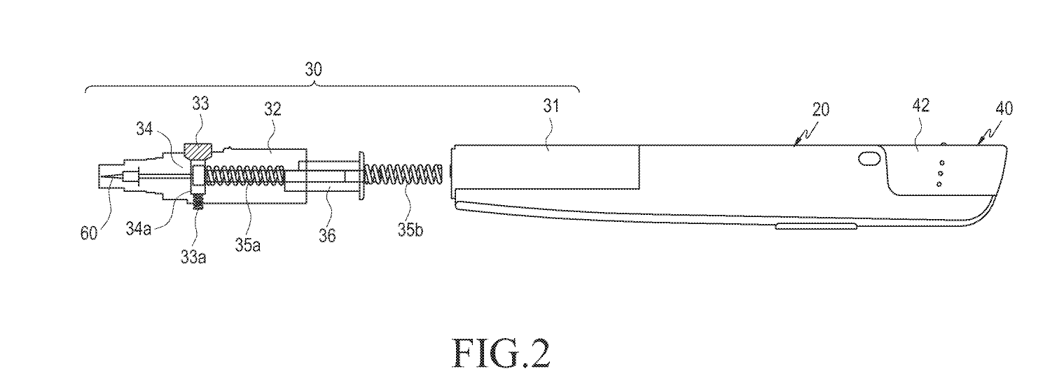

FIG. 2 is a sectional view illustrating an assembly of a lancing unit of a blood glucose meter according to an embodiment of the present disclosure.

Referring to FIGS. 1 and 2, the structure of the lancing unit 30 will be described. The lancing unit 30 includes a blood taking housing 31, a holder housing 32, a button 33, a holder 34, a plurality of elastic members 35, and a supporting member 36.

The blood taking housing 31 is formed at one side of the body 20 of the blood glucose meter and receives the holder housing 32, the button 33, the holder 34, the plurality of elastic members 35, and the supporting member 36. The holder housing 32 is received in the blood taking housing 31 such that the button 33 and the holder 34 are operated. The button 33 is received in the holder housing 32 so that a head portion of the button 33 is exposed to the outside of the blood taking housing 31 and pushed by a hand of the user. The holder 34 is provided with the lancet 60 and received in the holder housing 32. According to various embodiments of the present disclosure, the holder 34 may be toggled between a locked and unlocked state according to operation of the button 33. For example, the holder 34 is locked or unlocked as the button 33 is pushed, and returns the lancet 60 to an initial position or discharges the lancet 60 to the skin of the user in order to take blood. The elastic members 35 are coupled with the holder 34 and provide elasticity to the holder 34 so that the holder 34 can be moved. The supporting member 36 is coupled with the holder 34 so as to support the elastic members 35 when they are compressed and tensioned.

For example, the elastic members 35 include first and second elastic members 35a and 35b, in which the first elastic member 35a is compressed by the supporting member 36 and provides enough elasticity to discharge the holder 34 and the second elastic member 35b is tensioned by the supporting member 36 so that the holder 34 returns to the initial position after being discharged.

Furthermore, the holder 34 is preferably provided with a locking unit 34a which is latched by the button 33 or is released through a latching hole 33b formed in the button 33.

Hereinafter, an operation of the lancing unit 30 will be described in further detail.

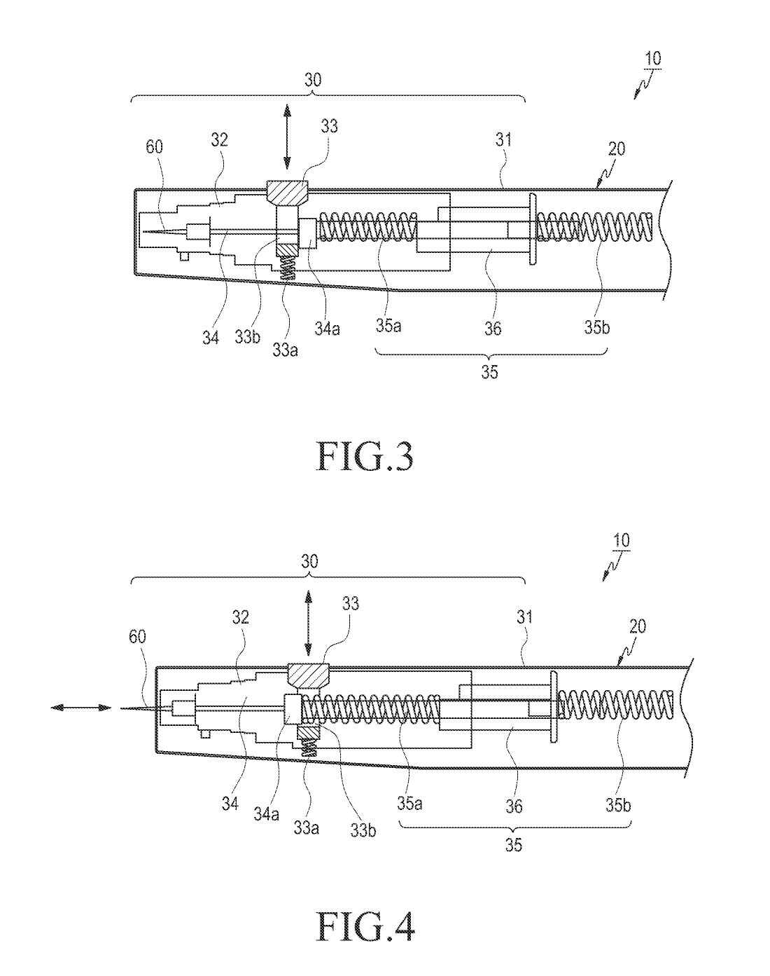

FIG. 3 is a sectional view illustrating a lancing unit of a blood glucose meter before operation of the lancing unit according to the embodiment of the present disclosure. FIG. 4 is a sectional view illustrating an operational state of a lancing unit of a blood glucose meter according to an embodiment of the present disclosure. For example, FIG. 4 is a sectional view illustrating an operational state of a lancing unit 30 after operating, in which the lancet 60 is discharged according to the embodiment of the present disclosure.

Referring to FIGS. 3 and 4, in the state that the holder housing 32 and the holder 34 are mounted in the blood taking housing 31, when the holder housing 32 and the holder 34 are pulled, the locking unit 34a of the holder 34 is moved and latched by the button 33 having the latching hole 33b. At this time, the button 33 penetrates the holder housing 32 and is exposed to the external environment of the blood taking housing 31. The first elastic member 35a is compressed by the supporting member 36 at the same time as the locking unit 34a of the holder 34 is latched by the button 33 having the latching hole 33b, and the second elastic member 35b provided to a rear portion of the supporting member 36 is simultaneously tensioned. In this state, when the user pushes the button 33 (e.g., with the user's hand), the button 33 with the latching hole 33b is moved downwardly so that the locking unit 34a of the holder 34 is separated from the button 33 and a lock of the locking unit 34a is released. Thereafter, the holder 34 is discharged by the first compressed elastic member 35a, and also the lancet 60 provided to the holder 34 is discharged along with the holder 34. The discharged lancet 60 penetrates the skin of the user so as to make blood flow. Thereafter, the second tensioned elastic member 35b contracts so as to enable the holder 34 to return to the initial position. At this time, the lancet 60 also returns to the initial position along with the holder 34.

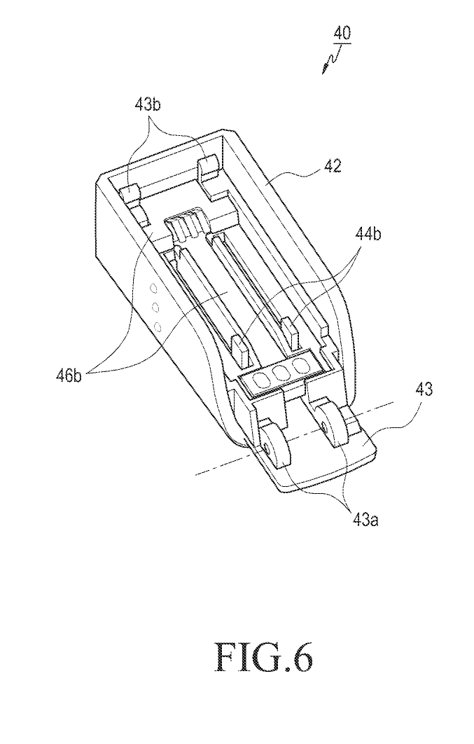

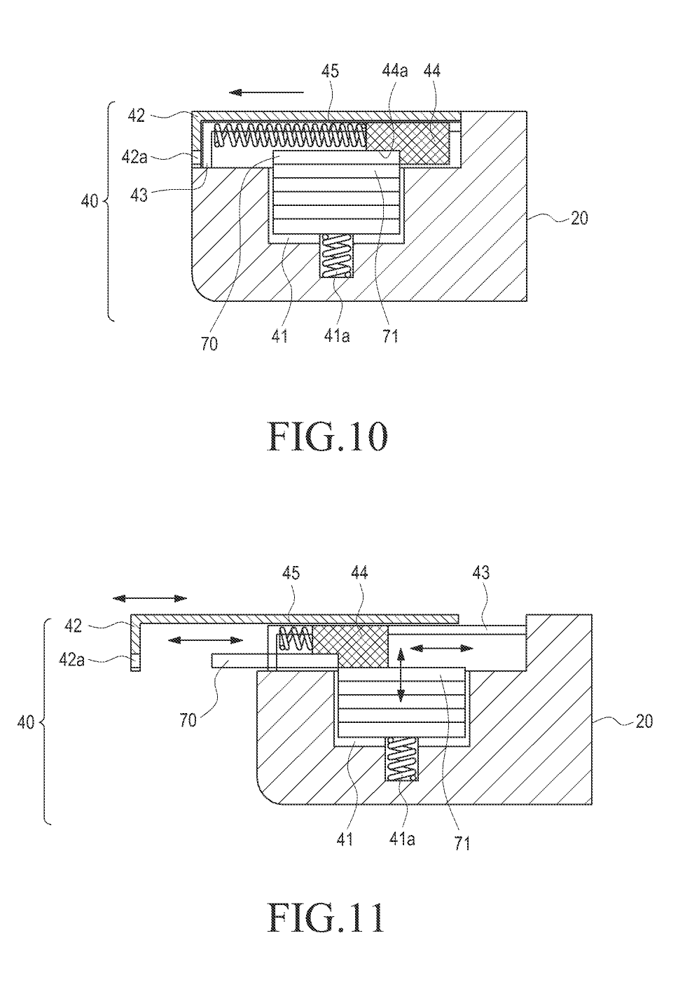

FIG. 6 is a perspective view illustrating an assembly of a sensor strip unit of a blood glucose meter according to an embodiment of the present disclosure. FIG. 10 is a sectional view illustrating a sensor strip unit of a blood glucose meter before a sensor strip operates according to an embodiment of the present disclosure. FIG. 11 is a sectional view illustrating a sensor strip unit of a blood glucose meter during an operation of a sensor strip unit according to an embodiment of the present disclosure.

Referring to FIGS. 5, 6, 10, and 11, the blood taken by the lancing unit 30 is introduced into the sensor strip 70 of the sensor strip unit 40 in order to test and/or measure the blood. For example, the sensor strip 70 may measure the glucose level of the blood. The sensor strip unit 40 includes a strip cartridge 41 having a plurality of sensor strips 70, a cover unit 42, a cover holder unit 43, a guide unit 44, an elastic member 45, and a guide holder unit 46. The strip cartridge 41 receives the sensor strips 70 which are stacked therein, and is provided at the other side of the body 20 of the blood glucose meter so as to push out the sensor strip 70 to the outside of the cover unit 42 as the guide unit 44 is slidably moved. The cover unit 42 is disposed on an upper portion of the strip cartridge 41 at the other side of the body 20 of the blood glucose meter, so as to slidably move and pivotally rotate. The cover unit 42 is coupled with and supported by the cover holder unit 43 so as to slidably move and rotate. The elastic member 45 is disposed in the cover holder unit 43 while being compressed and tensioned, in order to move the guide unit 44. The guide holder unit 46 is coupled with the guide unit 44 so as to support the movement of the guide unit 44.

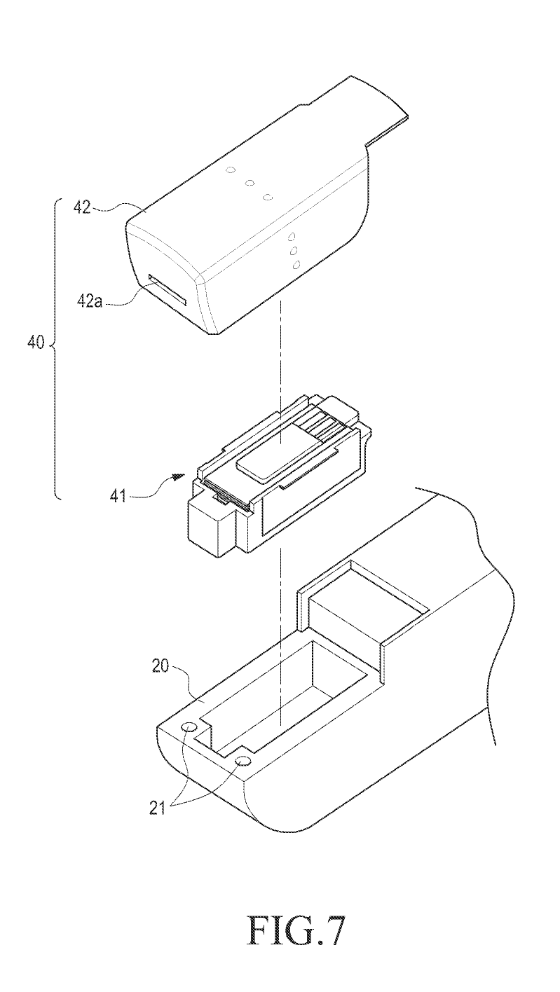

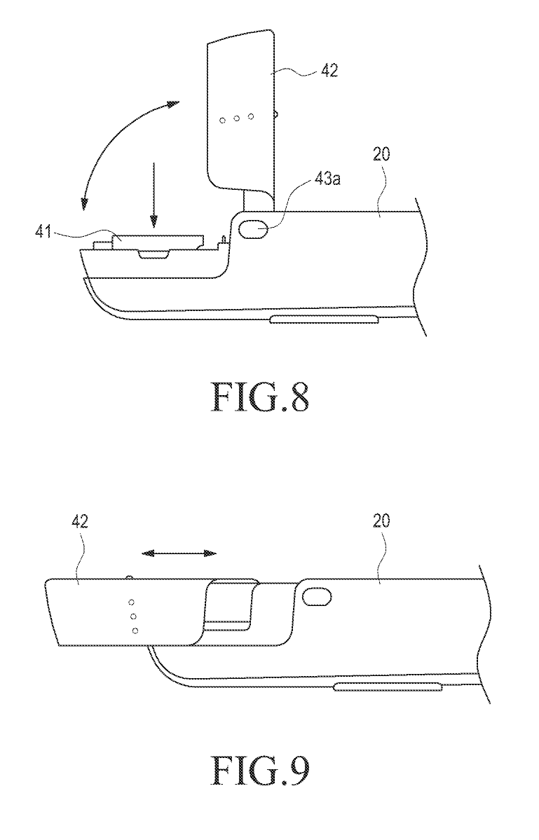

FIG. 7 is a perspective view illustrating an assembly of a sensor strip unit and a strip cartridge of a blood glucose meter according to an embodiment of the present disclosure. FIG. 8 is a side view illustrating a strip cartridge mounted on a blood glucose meter according to an embodiment of the present disclosure.

In addition, referring to FIGS. 6, 7 and 8, it is preferred that the cover holder unit 43 is provided with a holder hinge unit 43a around which the cover unit 42, the guide unit 44, the elastic member 45 and the guide holder unit 46 are pivotally rotated so as to open and close the blood glucose meter to mount and exchange the strip cartridge 41.

FIG. 13 is a perspective view illustrating a sensor strip of a blood glucose meter in which the sensor strip is exposed to an external environment according to an embodiment of the present disclosure.

Referring to FIG. 13, the cover unit 42 preferably has a thru-hole 42a formed therein, through which a sensor strip 70 may protrude out of the cover unit 42.

Referring to FIG. 5 again, the guide unit 44 has a strip coupling groove 44a which is engaged with and moved along with a sensor strip 70. Further, the guide holder unit 46 is preferably provided with a plurality of fixing portions 46b to fix the guide holder unit 46 to the cover unit 42. In addition, the guide holder 46 is preferably provided with a pair of guide grooves 46a with which a pair of guide protrusions 44b formed on the guide unit 44 is coupled to be slidably moved.

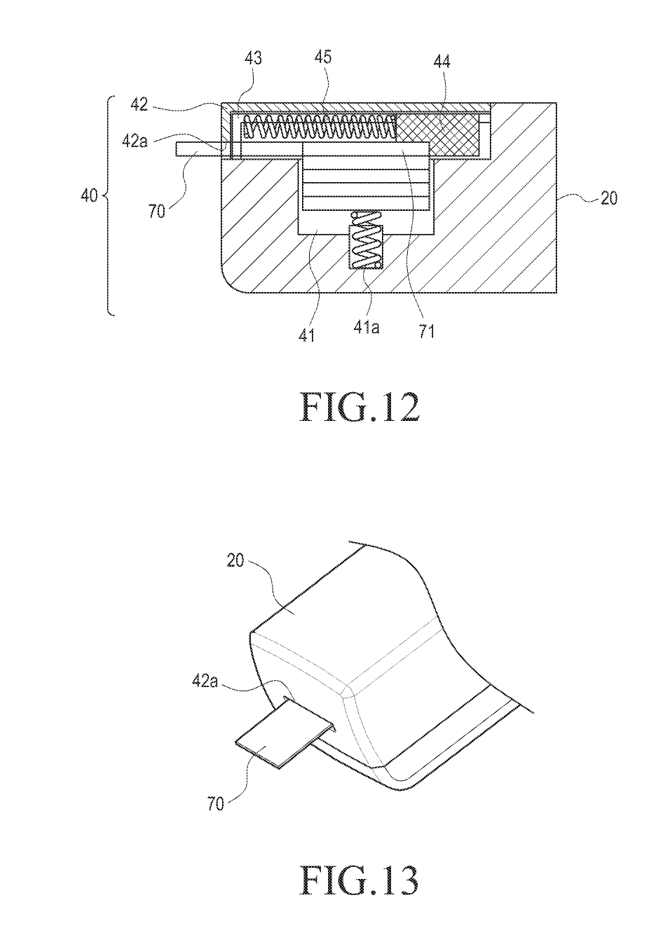

Hereinafter, an operation of the sensor strip unit 40 will be described with reference to FIGS. 9 and 12.

FIG. 9 is a side view illustrating a sensor strip unit of a blood glucose meter in which a cover of the sensor strip unit is slidably moved according to an embodiment of the present disclosure. FIG. 12 is a sectional view illustrating a sensor strip unit of a blood glucose meter after operation of the sensor strip unit according to an embodiment of the present disclosure.

For example, FIG. 9 is a side view illustrating a state that the cover unit 42 of the sensor strip unit 40 is slidably moved according to the embodiment of the present disclosure, FIG. 10 is a sectional view illustrating the sensor strip unit 40 of the blood glucose meter before operating according to the embodiment of the present disclosure, FIG. 11 is a sectional view illustrating the cover unit 42 of the sensor strip unit 40 of the blood glucose meter according to the embodiment of the present disclosure, in which the cover unit 4 is slidably moved, and FIGS. 12 and 13 are views illustrating the sensor strip 70 protruding from the cover unit 42 after the sensor strip unit 40 operates according to the embodiment of the present disclosure.

Referring to FIGS. 10 and 11, the user pushes and slidably moves the cover unit 42 of the sensor strip unit 40 in a direction from the body 20 to the external environment of the blood glucose meter, in order to measure the glucose level of the blood taken from the skin of the user by using the lancing unit 30.

As described above, as illustrated in FIG. 11, the guide unit 44 is moved along with the cover unit 42, while moving one sensor strip 70 disposed at an upper end of the strip cartridge 41. Because the guide unit 44 has the strip coupling groove 44a which is engaged with and moves along with the sensor strip 70, one sensor strip 70 moves along with the guide unit 44 when the guide unit 44 is slidably moved. At this time, the guide unit 44 slides along the pair of guide grooves 46a formed in the guide holder unit 46.

Referring to FIG. 11, further, the elastic member 45 provided between the cover holder unit 43 and the guide unit 44 is compressed. In this state, when the user releases the cover unit 42, the compressed elastic member 45 enables the guide unit 44 to slidably move to the body 20 of the blood glucose meter while enabling the cover unit 42 to slidably move along with the guide unit 44 in relation to the body 20 of the blood glucose meter.

At this time, referring to FIGS. 12 and 13, a next sensor strip 71 is elevated in the strip cartridge 41 and is engaged with the strip coupling groove 44a of the guide unit 44. The next sensor strip 71 is elevated by a spring 41a provided at a lower portion of the strip cartridge 41 and simultaneously engaged with the strip coupling groove 44a. The next sensor strip 71 supports the movement of the one sensor strip 70 which is previously and slidably moved. As described above, the sensor strip 70 protrudes through the thru-hole 42a of the cover unit 42 and is maintained in an outwardly protruding state.

In this state, referring to FIG. 14, the measurement unit 50 is preferably provided with a plurality of operational buttons 51 for operating the sensor strip 70, and a display unit 52.

According to various embodiments of the present disclosure, when blood including glucose is introduced into the sensor strip 70, an enzyme reaction layer dissolves the blood and reacts with an enzyme in the blood so as to oxidize the glucose. As a result, an oxidizing agent is deoxidized. The measurement unit 50 illustrated in FIG. 14 measures an anodic current obtained by electrochemically oxidizing the oxidizing agent which is deoxidized. The display unit 52 displays a result of the measurement unit. For example, the display unit 52 displays the result of the measuring of the anodic current obtained by electrochemically oxidizing the oxidizing agent which is deoxidized. For example, the display unit 52 illustrated in FIG. 14 simultaneously or contemporaneously displays the measured value.

Hereinafter, an assembling of the lancing unit 30 provided at one side of the blood glucose meter 10 will be described in more detail with reference to FIGS. 2 and 3. First, in the structure of the lancing unit 30 provided at the one side of the blood glucose meter 10, the holder housing 32 is coupled with the blood taking housing 31 disposed on a portion of the body 20 of the blood glucose meter. In this state, the button 33 is assembled with the holder housing 32 so as to be simultaneously exposed to the external environment of the blood taking housing 31. The holder unit 34 is assembled with the holder housing 32. At this time, the holder unit 34 is latched by the button 33 having the latching hole 33b. For example, the locking unit 34a of the holder unit 34 passes through the latching hole 33b and is latched by the button 33. The holder unit 34 is inserted in the first elastic member 35a, and then is coupled with the supporting member 36. The supporting member 36 is coupled with the second elastic member 35b at a rear surface thereof The button 33 has a button spring 33a disposed at a lower portion thereof, which enables the button 33 to return to an initial position when the button is pushed.

An assembly of the sensor strip unit 40 provided at the other side of the blood glucose meter will be described in detail with reference to FIGS. 5 to 7. First, the cover unit 42 is coupled with the holder unit 43 so as to be slidably moved along the holder unit 43 and to pivotally rotate along with the holder unit 43. The guide unit 44 is assembled with the cover unit 42, which is slidably moved and pivotally rotated along with the cover unit 42. The elastic member 45 is disposed between the cover holder unit 43 and the guide unit 44. In this state, the guide holder unit 46 supporting the movement of the guide unit 44 is coupled with the guide unit 44. Referring to FIGS. 7 and 8, the strip cartridge 41 provided with the plurality of the sensor strips 70 is mounted on the body 20 of the blood glucose meter. The sensor strip unit 40 assembled as described above is provided on an upper portion of the strip cartridge 41.

As described above, in the pen typed blood glucose meter, the lancing unit 30 and the sensor strip unit 40 are assembled with the body 20 at both ends of the body 20, respectively. The measurement unit 50 is embedded in the body 20 of the blood glucose meter.

In other words, when the holder unit 34 provided at the one side of the body 20 of the blood glucose meter is pulled out, the locking unit 34a of the holder unit 34 is moved and latched by the button 33 with the latching hole 33b. In this state, when the user pushes the button 33 with the user's hand, the button 33 with the latching hole 33b is moved downwardly so that the locking unit 34a of the holder unit 34 is separated from the button 33 and a lock of the locking unit 34a is released. As a result, the holder unit 34 is discharged by the first compressed elastic member 35a. At this time, the lancet 60 is discharged along with the holder unit 34, and penetrates the skin of the user so that the blood flows out. The blood may be applied and/or absorbed by the sensor strip 70 protruding from the cover unit 42.

In other words, when the cover unit 42 is slidably moved outwardly from the body 20 of the blood glucose meter, the guide unit 44 is moved along with the cover unit 42 and moves one sensor strip 70 arranged on an upper end of the strip cartridge 41, so that the sensor strip 70 protrudes out of the cover unit 42. At this time, the elastic member 45 provided between the cover holder unit 43 and the guide unit 44 is compressed. In this state, when the user releases the cover unit 42, the compressed elastic member 45 enables the guide unit 44 to slidably move to the body 20 of the blood glucose meter while enabling the cover unit 42 to slidably move along with the guide unit 44 to the body 20 of the blood glucose meter. The sensor strip 70 which is slidably moved protrudes through the thru-hole 42a to the external environment of the cover 42.

Referring to FIGS. 13 and 14, when the taken blood is introduced to the sensor strip 70 protruding from the cover unit 42, the sensor strip 70 electrochemically reacts with the blood. Thereafter, the glucose measurement unit 50 measures the blood. For example, the glucose measurement unit 50 measures the electrochemical reaction with the blood. As an example, the glucose measurement unit 50 receives electric signals from the plurality of electrodes (not shown) contacting the sensor strip 70, measures the glucose level of the blood using the electric signals. Thereafter, the display unit 52 may display a result of the measurement made in relation to the blood sample applied to the sensor strip 70. For example, the display unit 52 displays a measured value.

The blood glucose meter according to the related art has a disadvantage in that a sensor strip may be polluted or contaminated as a user directly holds the sensor strip with the user's hand during the measurement of the blood glucose.

In order to overcome the disadvantage of the related art associated with potentially polluting or contaminating the sensor strip during measurement of the blood, the sensor strip according to various embodiments of the present disclosure is configured to protrude out of the cover unit as a user slidably moves the cover unit instead of holding the sensor strip. Accordingly, preventing the sensor strip from being polluted by alien (e.g., foreign) substances on the hand of the user may be prevented.

According to various embodiments of the present disclosure, the measurement unit 50 illustrated in FIG. 14 may be provided with an amplifying unit (not shown). When electricity is applied to an operational electrode, the amplifying unit detects the amount of electric current applied to the operational electrode and outputs a value of an electric voltage. According to various embodiments of the present disclosure, the blood glucose meter may include a controller (not shown). Further, the measurement unit may be provided with an A/D converter. The A/D converter converts an analog value of the voltage output from the amplifying unit into a digital signal, and transmits the digital signal to a controller. The controller controls a whole operation of the measurement unit 50 illustrated in FIG. 14, and displays, on the display unit 52, a value of the blood glucose, which is finally measured. The operational electrode, the A/D converter, the amplifying unit and the controller described above are not shown.

According to the related art, because a user always carries a plurality of units such as a portable measurement unit, a sensor strip, a lancet, a lancing unit, and the like in order to measure the user's blood glucose, the user may be inconvenienced when measuring the blood glucose of the user.

In order to overcome the disadvantage of the blood glucose meter according to the related art, the blood glucose meter according to various embodiments of the present disclosure is configured to have the structure in that the body 20 illustrated in FIG. 2, the lancing unit 30 having the lancet illustrated in FIG. 1, and the sensor strip unit 40 illustrated in FIG. 5 are integrated with the measurement unit 50 illustrated in FIG. 14. Accordingly, the user may always carry the above-mentioned units and may use the blood glucose meter with one hand, thereby improving usability of the blood glucose meter.

Furthermore, the measurement unit is configured to have the lancing unit 30 illustrated in FIG. 1 which includes the lancet 60 to take blood and is disposed at one side of the body 20 of the measurement unit 50 illustrated in FIG. 14 and the sensor strip unit 40 illustrated in FIG. 5 which reacts with the blood and is disposed at the other side of the body 20. Accordingly, it is possible to remove inconvenience caused by an arrangement in which the lancing unit and the sensor strip unit are disposed at one side of the blood glucose meter according to the related art, and also to exactly introduce the blood into the sensor strip. Further, the lancing unit can be prevented from being polled by the blood, and usability of a product can be improved.

Referring to FIGS. 7 and 8, the body 20 of the blood glucose meter 20 has one or more coupling grooves 21 with which the plural coupling protrusions 43b illustrated in FIG. 5 formed on the cover holder unit 43 are engaged in order to fix the cover holder unit 43 to the body 20 when the cover unit 42 is slidably moved, and are separated from in order that the cover holder unit 43 pivotally rotates along with the cover unit 42 when the cover unit 42 pivotally rotates.

For example, as illustrated in FIGS. 6, 7, and 8, in order to exchange the strip cartridge 41, the coupling protrusions 43b of the cover holder unit 43 are separated from the coupling grooves 21 of the body 20 of the blood glucose meter, and pivotally rotate along the cover unit 42. At this time, the strip cartridge 41 which is previously empty is removed from the body 20 of the blood glucose meter which is open, and a new strip cartridge is mounted on the body 20 of the blood glucose meter. Thereafter, the cover unit 42 and the cover holder unit 43 are enabled to rotate. At this time, the coupling protrusions 43b of the cover holder unit 43 are coupled with the coupling grooves 21 respectively again. In this state, when the cover unit 42 is slidably moved, the cover holder unit 43 is held in a fixed state.

Referring to FIGS. 10, 11 and 12, the sensor strips 70 are stacked in the strip cartridge 41, and sequentially elevated and discharged from the strip cartridge 41 when the cover unit is slidably moved.

For example, as illustrated in FIG. 12, when the cover 42 is slidably moved, the guide unit 44 moves along with the cover unit 42 so as to slidably move and discharge the sensor strip 70, which is stacked and disposed at an upper end of the strip cartridge 41, to the outside of the blood glucose meter through the thru-hole 42a of the cover unit 42. At this time, another sensor strip 71 illustrated in FIG. 12 stacked under the discharged sensor strip 70 is elevated and engaged with the guide unit 44 in preparation for discharge.

As described above, the sensor strips 70 and 71 are stacked up, and sequentially elevated and discharged as the cover unit 42 is slidably moved.

Various embodiments of the present disclosure are described in relation to a blood glucose meter. However, the blood glucose meter according the various embodiments of the present disclosure is used as a representative example to measure the blood glucose of the user. Various embodiments of the present disclosure are not limited to the measurement of the blood glucose, and also can be applied to electrochemical test apparatuses, for example, a portable test apparatus and the like, in various forms to take and analyze blood.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. A blood glucose meter comprising:
a measurement unit integrally assembled with a body of the blood glucose meter, and including a lancing unit having a lancet and a sensor strip unit.

2. A blood glucose meter comprising:
a body of the blood glucose meter;
a lancing unit which is provided at one side of the body of the blood glucose meter and includes a lancet to puncture a skin in order to sample blood of a user;
a sensor strip unit which is provided at another side of the body of the blood glucose meter which is different than the one side of the body at which the lancing unit is provided, the sensor strip unit being configured to sample the blood of the user; and
a measurement unit which measures the blood sampled by the sensor strip unit.

3. The blood glucose meter of claim 2, wherein the body of the blood glucose meter is a pen type.

4. The blood glucose meter of claim 2, wherein the lancing unit comprises:
a blood taking housing disposed at the one side of the body of the blood glucose meter;
a holder housing which is disposed in the blood taking housing;
a button which is provided to the holder housing and exposed to an external environment of the blood taking housing;
a holder unit which is provided in the holder housing, which has the lancet, and which is latched or released as the button is pushed while being moved to discharge or return the lancet to an initial position;
a plurality of elastic members which are coupled with the holder unit and provide elasticity to the holder unit so as to move the holder unit; and
a support member which is coupled with the holder unit to compress and tension the elastic members.

5. The blood glucose meter of claim 4, wherein the holder unit further comprises:
a locking unit latched by or released from the button, and
wherein the button comprises a latching hole.

6. The blood glucose meter of claim 4, wherein the elastic members comprise:
a first elastic member which is compressed by the supporting member and provides elasticity to the holder unit in order to discharge the holder unit; and
a second elastic member which is tensioned by the supporting member in order to return the holder unit to an initial position after the holder unit is discharged.

7. The blood glucose meter of claim 4, further comprising:
a button spring which is disposed under the button and which biases the button to an initial position when the button is pushed.

8. The blood glucose meter of claim 2, wherein the sensor strip unit comprises:
a strip cartridge which is provided at another side of the body of the blood glucose meter which is different than the one side of the body at which the lancing unit is provided, the strip cartridge comprising a plurality of sensor strips;
a cover unit which is disposed on the strip cartridge and which is slidably moved and pivotally rotated at the corresponding side of the body of the blood glucose meter;
a cover holder unit which is coupled with the cover unit and which supports the cover unit so that the cover unit is slidably moved and pivotally rotated;
a guide unit which is coupled with the cover unit and guides a movement of the sensor strip when being slidably moved along with the cover unit, so that the sensor strip protrudes out of the cover unit;
an elastic member which is disposed between the cover holder unit and the guide unit and is compressed and tensioned so as to move the guide unit; and
a guide holder unit which is coupled with the guide unit so as to support a movement of the guide unit.

9. The blood glucose meter of claim 8, wherein the cover holder unit further comprises:
a holder hinge unit around which the cover unit pivotally rotates to open and close the body of the blood glucose meter, so as to mount and exchange the strip cartridge.

10. The blood glucose meter of claim 8, wherein the cover unit further comprises:
a thru-hole through which the sensor strip protrudes to the outside.

11. The blood glucose meter of claim 8, wherein the guide unit further comprises:
a strip coupling groove which is engaged with the sensor strip so as to slidably move the sensor strip.

12. The blood glucose meter of claim 8, wherein the guide holder unit further comprises:
a plurality of fixing units to fix the guide holder unit to the cover unit.

13. The blood glucose meter of claim 8, wherein the guide holder unit further comprises:
a pair of guide protrusions formed on the guide unit and a pair of guide grooves in which the guide protrusions are slidably inserted.

14. The blood glucose meter of claim 10, wherein when the sensor strip is protrudes from the cover unit, the cover unit is slidably moved in a protruding direction and the guide unit is moved along with the cover unit so as to move the sensor strip of the strip cartridge, and the guide elastic member is compressed, while when the cover unit is released after the sliding movement of the cover unit, the compressed guide elastic member is tensioned so as to slidably move the cover unit and the guide unit in a direction toward the body of the blood glucose meter and to make the moved sensor strip to protrude through the thru-hole of the cover unit, and also as the cover unit and the guide unit return to the initial position, another sensor strip provided to the strip cartridge is elevated by a spring provided at a lower portion of the strip cartridge and engaged with the guide unit.

15. The blood glucose meter of claim 2, wherein the measurement unit comprises:
a plurality of operation buttons and a display unit.

16. The blood glucose meter of claim 8, wherein the body of the blood glucose meter has one or more coupling grooves which are coupled with plural coupling protrusions formed on the cover holder unit so as to fix the cover holder unit to the cover unit when the cover unit is slidably moved, while being separated from the plural coupling protrusions of the cover holder unit so that the cover unit rotates along with the cover holder unit when the cover unit is pivotally rotated.

17. The blood glucose meter of claim 8, wherein the sensor strips are stacked in the strip cartridge in order to sequentially move and discharge the sensor strip from the strip cartridge.

18. A blood glucose meter comprising:
a body of the blood glucose meter;
a lancing unit configured to puncture a skin of a user;
a sensor strip unit comprising a plurality of sensor strips, the sensor strip unit configured to discharge one of the plurality of sensor strips in order to sample the blood of the user; and
a measurement unit configured to measure the sampled blood of the user.

19. The blood glucose meter of claim 18, further comprising:
a display unit configured to display results of the measurements made on the sampled blood of the user.

20. The blood glucose meter of claim 19, wherein the lancing unit, the sensor strip unit, the measurement unit, and the display unit are integrated into the body of the blood glucose meter.
